(19) United States
(12) Patent Application Publication
(10) Pub. No.: US 2026/0046958 A1
(43) Pub. Date: Feb. 12, 2026

(54) MEASURING DEVICE

(71) Applicant: OMRON HEALTHCARE Co., Ltd., Kyoto (JP)

(72) Inventors: Tetsuzo OKAZAKI, Kyoto (JP); Yasunori FUKUDA, Kyoto (JP)

(21) Appl. No.: 19/363,159

(22) Filed: Oct. 20, 2025

Related U.S. Application Data
(63) Continuation of application No. PCT/JP2024/000653, filed on Jan. 12, 2024.

(30) Foreign Application Priority Data
Jun. 7, 2023 (JP) 2023-093953

(51) Int. Cl. H04W 76/14; A61B 5/00; A61B 5/022; H04B 17/318; H04W 16/28

(52) U.S. Cl. CPC H04W 76/14; A61B 5/02225; A61B 5/742; H04B 17/328; H04W 16/28

(57) ABSTRACT

A measuring device capable of performing pairing with an intended information terminal. A blood pressure monitor includes: a communication unit configured to communicate by a communication method that enables pairing; a processor; and a housing adapted to accommodate the processor and the communication unit. An antenna of the communication unit has directivity in a first direction that is larger than directivity in a second direction other than the first direction and is largest. An information display section from which first information can be acquired by imaging is provided on a side surface among outer surfaces of the housing, the side surface including an intersection with a virtual line extending from the antenna in the first direction. When the processor receives a signal including information based on the first information and a received strength of the signal is at a threshold or greater, the processor starts processing necessary for pairing.

### CROSS-REFERENCE TO RELATED APPLICATIONS

This application is the U.S. national stage application filed pursuant to 35 U.S.C. 365 (c) and 120 as a continuation of International Patent Application No. PCT/JP2024/000653, filed Jan. 12, 2024, which application claims priority to Japanese Patent Application No. 2023-093953, filed Jun. 7, 2023, which applications are incorporated herein by reference in their entireties.

### TECHNICAL FIELD

The present invention relates to a measuring device.

### BACKGROUND ART

A measuring device described in the present specification includes a biological information measuring device configured to measure biological information such as a weight, a body composition, blood pressure, a pulse, heartbeat, a body temperature, blood glucose, or a blood oxygen saturation level, and an activity amount measuring device configured to measure an activity amount such as the number of steps, a walking distance, or calorie consumption. The measuring device includes a measuring sensor for measuring a measurement target amount. The measurement target amount of the measuring sensor includes biological information such as a weight, a body fat percentage, a blood pressure value, a pulse rate, a heart rate, a body temperature, a blood glucose level, or a blood oxygen saturation level, and an activity amount such as the number of steps, a walking distance, or calorie consumption, depending on the measuring device. A measurement result of such a measuring device is recorded and analyzed by an information terminal such as a smartphone, a tablet terminal, a laptop computer, and a desktop personal computer.

When such a measurement result is recorded and analyzed, it is desirable that the information terminal be able to automatically acquire the measurement result from the measuring device, instead of having a user input the measurement result to the information terminal each time. Specifically, for example, a method in which the measurement result from the measuring device is transferred to the information terminal with the use of near-field wireless communication such as Bluetooth (registered trademark) is conceivable.

Patent Document 1 describes a wearable device capable of measuring biological information and wirelessly transmitting the biological information to an external device.

Patent Document 2 describes a biological information measuring device configured to communicate with an external terminal by ultrasonic waves, the biological information measuring device including: communication means configured to communicate information including measurement information with the external terminal; and light projection means configured to visualize information about a place where the external terminal is to be placed, by casting light.

### CITATION LIST

### Patent Literature

Patent Document 1: JP 04665904 B
Patent Document 2: JP 2020-156600 A

### SUMMARY OF INVENTION

### Technical Problem

In order to perform secure near-field wireless communication between a measuring device and an information terminal, processing of pairing the measuring device and the information terminal is required. Pairing refers to processing of sharing encryption information used for near-field wireless communication between the measuring device and the information terminal. When pairing is performed, it is necessary to prevent pairing with an unintended information terminal.

An object of the technology of the present disclosure is to provide a measuring device capable of performing pairing with an intended information terminal.

### Solution to Problem

The technology of the present disclosure is described below. Note that components and the like corresponding to those in the following embodiments are indicated in parentheses, but the components are not limited thereto.

(1) A measuring device (blood pressure monitor 10), including:
a near-field wireless communication unit (communication unit 12) configured to perform communication by a communication method that enables pairing;
a processor (processor 11); and a housing (housing 17) adapted to accommodate the processor and the near-field wireless communication unit, wherein an antenna (antenna 12A) of the near-field wireless communication unit has directivity in a first direction that is larger than directivity in a second direction other than the first direction and is largest, an information display section (information display section 17E) from which first information can be acquired by imaging is provided on a surface (side surface 17C) among outer surfaces of the housing, the surface including an intersection (intersection P) with a virtual line extending from the antenna in the first direction, and when the processor receives a signal including information based on the first information and a received strength of the signal is at a threshold or greater, the processor starts processing necessary for pairing.

According to (1), when pairing is performed between an information terminal and the measuring device, the information display section is imaged by using the information terminal to acquire the first information, and the signal including the information based on the first information is transmitted from the information terminal to the measuring device. Thus, the signal from the information terminal is received by the measuring device in a state where the information terminal is located in a direction in which the directivity of the antenna is high and at a position near the measuring device. As a result, in the measuring device, the received strength of the signal becomes sufficiently large, and thus the information terminal that is a transmission source of the signal can be specified as a pairing partner. Accordingly, since the information terminal located near the measuring device and at the position in which information can be acquired from the information display section can be specified as the pairing partner, pairing with the intended information terminal can be realized.

(2) The measuring device according to (1), wherein when the processor receives the signal and the received strength of the signal is at the threshold or greater, the processor starts transmission of a broadcast signal including information necessary to establish a connection for communication by near-field wireless communication (step S14), establishes a connection with an information terminal that has responded to the broadcast signal (step S20), and performs pairing.

According to (2), by only imaging the information display section with the information terminal and transmitting the signal from the information terminal to the measuring device, the measuring device can be shifted to the state of transmitting the broadcast signal. Accordingly, a state in which the measuring device always transmits a broadcast signal can be avoided, and thus power consumption of the measuring device can be reduced. In addition, a user of the measuring device can perform pairing between the measuring device and the information terminal without confusion.

(3) The measuring device according to (2), wherein when the processor receives the signal and the received strength of the signal is at the threshold or greater, the processor reduces a radio signal strength of the antenna to be lower than a radio signal strength at a timing before the signal is received.

According to (3), when there is a different information terminal located at a position more or less separated from the measuring device and located on a virtual line, the radio signal strength from the measuring device becomes weak at the different information terminal. As a result, connection establishment between the different information terminal and the measuring device can be prevented. Therefore, pairing with the intended information terminal located near the measuring device can be realized.

(4) The measuring device according to any one of (1) to (3), wherein the information display section is a display (display section 15) on which the first information can be displayed, and the display is configured to display a measurement result.

According to (4), since the display that displays the measurement result and the information display section can be shared, it is not necessary to add a dedicated information display section, allowing for cost reduction and improved design of the device. Further, as long as the display operates, the information can be read from the information display section, and thus a lack of information can be prevented. Furthermore, the information display section cannot be easily viewed by others, and security can be improved.

### Advantageous Effects of Invention

According to the technology of the present disclosure, the measuring device capable of performing pairing with an intended information terminal can be provided.

### BRIEF DESCRIPTION OF DRAWINGS

Various embodiments are disclosed, by way of example only, with reference to the accompanying schematic drawings in which corresponding reference symbols indicate corresponding parts, in which:

FIG. 1 is a schematic diagram illustrating a schematic configuration of a management system 100.

FIG. 2 is a schematic diagram illustrating an example of an external configuration of a blood pressure monitor 10 illustrated in FIG. 1.

FIG. 3 is a diagram illustrating a schematic configuration of a communication unit 12, and is a schematic diagram of a housing 17 as viewed from a rear surface 17A side.

FIG. 4 is a diagram illustrating the schematic configuration of the communication unit 12, and is a schematic diagram of the housing 17 as viewed from an upper surface 17B side.

FIG. 5 is a diagram of the housing 17 of the blood pressure monitor 10 as viewed from a side surface 17C side on which an information display section 17E is provided.

FIG. 6 is a sequence chart illustrating a method of pairing between the blood pressure monitor 10 and a smartphone 20.

### DESCRIPTION OF EMBODIMENTS

Overview of Measuring Device of Technology of the Present Disclosure A measuring device of the technology of the present disclosure includes a near-field wireless communication unit configured to perform communication by a communication method (for example, Bluetooth (registered trademark)) that enables pairing, a processor, and a housing adapted to accommodate the processor and the near-field wireless communication unit. An antenna of the near-field wireless communication unit has directivity in a first direction that is larger than directivity in a second direction other than the first direction and is largest, an information display section from which first information can be acquired by imaging is provided in a region, on an outer surface of the housing, including an intersection with a virtual line extending from the antenna in the first direction. When the processor receives a signal including information based on the first information and a received radio signal strength of the signal is at a threshold or greater, the processor starts processing necessary for pairing. According to this configuration, when pairing is performed between an information terminal and the measuring device, the information display section is imaged by using the information terminal to acquire the first information, and the signal including the information based on the first information is transmitted from the information terminal to the measuring device. Thus, the signal from the information terminal is received by the measuring device in a state where the information terminal is located in a direction in which the directivity of the antenna is high and at a position near the measuring device. As a result, in the measuring device, the received radio signal strength of the signal becomes sufficiently large, and thus the information terminal as a transmission source of the signal can be specified as a pairing partner. Accordingly, since the information terminal located near the measuring device and at the position in which information can be acquired from the information display section can be specified as the pairing partner, pairing with the intended information terminal can be realized.

Hereinafter, a configuration example of a management system 100 including a measuring device of the present embodiment will be described.

### System Configuration

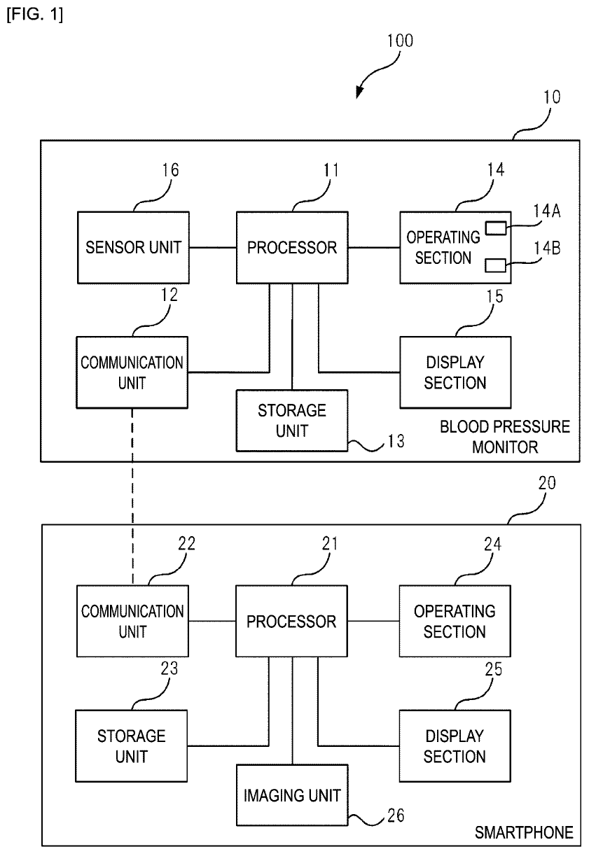

FIG. 1 is a schematic diagram illustrating a schematic configuration of the management system 100. The management system 100 includes a blood pressure monitor 10, which is an example of the measuring device, and a smartphone 20, which is an example of the information terminal, and is a system for managing measurement data, a program, and the like of the blood pressure monitor 10 by using the smartphone 20. The blood pressure monitor 10 and the smartphone 20 are configured to be able to communicate with each other by near-field wireless communication. A method that enables pairing can be adopted as the near-field wireless communication method. The following description assumes the near-field wireless communication method to be Bluetooth (registered trademark). Hereinafter, an owner who owns both the blood pressure monitor 10 and the smartphone 20 is referred to as a user.

### Measuring Device

The blood pressure monitor 10 includes a processor 11, a communication unit 12, a storage unit 13, an operating section 14, a display section 15, and a sensor unit 16.

The sensor unit 16 includes a pressure sensor disposed as a measuring sensor in a cuff portion of the blood pressure monitor 10, and detects, by the pressure sensor, a pulse wave from a blood vessel of the user under appropriate cuff pressure. The blood pressure monitor 10 can calculate blood pressure information including systolic blood pressure, diastolic blood pressure, and a pulse based on the pulse wave detected by the sensor unit 16.

The communication unit 12 is a communication interface for performing near-field wireless communication, and includes a communication antenna and various circuits.

The storage unit 13 includes, for example, a non-transitory storage medium such as a flash memory in addition to a working memory such as a random access memory (RAM). A variety of information such as measured blood pressure information are stored in this storage medium.

The operating section 14 is input means such as a button or a touch panel that receives an input from the user, and accepts various operations such as ON/OFF of a power supply, a start of measurement of blood pressure information, and a selection of an item from the user. The operating section 14 includes a measurement start button 14A for providing an instruction to start the measurement of blood pressure information, and a communication button 14B for operating the communication unit 12 (enabling near-field wireless communication). The measurement start button 14A and the communication button 14B may be hardware buttons, or may be software buttons displayed on the display section 15 equipped with a touch panel.

The display section 15 includes, for example, a display such as an organic electro-luminescence (EL) display or a liquid crystal display, and displays the measured blood pressure information or the like.

The processor 11 comprehensively controls each unit of the blood pressure monitor 10. The processor 11 is, for example, a central processing unit (CPU) that is a general-purpose processor configured to execute software (program) and perform various functions, a programmable logic device (PLD) that is a processor whose circuit configuration can be changed after manufacturing, such as a field programmable gate array (FPGA), or a dedicated electric circuit that is a processor including a circuit configuration specifically designed to execute specific processing, such as an application specific integrated circuit (ASIC). The processor 11 may include one processor, or may include a combination of the same type or different types of two or more processors (for example, a plurality of FPGAs or a combination of a CPU and an FPGA). More specifically, the hardware structure of the processor 11 is an electric circuit (circuitry) in which circuit elements such as semiconductor elements are combined.

Upon detecting pressing of the measurement start button 14A included in the operating section 14, the processor 11 accepts an instruction to start measurement, supplies air to a cuff, and calculates blood pressure information based on a pulse wave detected by the sensor unit 16 under appropriate cuff pressure. Then, the processor 11 displays the calculated blood pressure information on the display section 15. The processor 11 controls each component of the blood pressure monitor 10 so as to execute processing according to an operation performed by the user via the operating section 14. The action of pressing the measurement start button 14A constitutes an operation of starting operation of the sensor unit 16. By being pressed only once in a short time (performing only a so-called short press operation) instead of being continuously pressed for a predetermined time (performing a so-called long press operation), the measurement start button 14A can input the instruction to start measurement to the processor 11.

Upon detecting that the communication button 14B included in the operating section 14 is pressed, the processor 11 activates the communication unit 12. The pressing operation of the communication button 14B necessary for activating the communication unit 12 is, for example, a long press operation.

In FIG. 1, the blood pressure monitor 10 is illustrated as an example of the measuring device, but the blood pressure monitor 10 can be replaced with a scale, a body composition meter, a pulse meter, a heart rate meter, a thermometer, a blood glucose meter, a pulse oximeter, an activity meter, or the like. In any of these measuring devices, the sensor unit 16 includes various measuring sensors (a pressure sensor, a pulse wave sensor, a blood glucose sensor, a photoelectric sensor, a temperature sensor, an acceleration sensor, or the like) for measuring a physical quantity of a measurement target. In a case where the measuring device is a biological information measuring device, upon detecting that the measurement start button 14A included in the operating section 14 is pressed, the processor 11 operates a measuring sensor (a pressure sensor, a pulse wave sensor, a blood glucose sensor, a photoelectric sensor, a temperature sensor, or the like) included in the sensor unit 16 to measure biological information. In a case where the measuring device is an activity amount measuring device, when a power button for turning on the device is pressed instead of the measurement start button 14A, the processor 11 activates a measuring sensor (an acceleration sensor, an angular velocity sensor, or the like) included in the sensor unit 16 to measure an activity amount.

### Information Terminal

The smartphone 20 includes a processor 21, a communication unit 22, a storage unit 23, an operating section 24, a display section 25, and an imaging unit 26.

The communication unit 22 is a communication interface for performing near-field wireless communication, and includes a communication antenna and various circuits.

The storage unit 23 includes a non-transitory storage medium such as a flash memory in addition to a working memory such as a RAM. The storage medium stores a variety of information including application programs (an operation program of the information terminal, a management application described below).

The operating section 24 is input means such as a button or a touch panel that accepts an input from a user, and accepts various operations from the user.

The display section 25 includes, for example, a display such as an organic EL display, a liquid crystal display, or the like.

The imaging unit 26 includes an imaging element and an image processing unit, and captures an image of a subject by the imaging element to generate captured image data.

The processor 21 comprehensively controls each unit of the smartphone 20. Similarly to the processor 11, the processor 21 includes one or a plurality of processors. The processor 21 executes various programs stored in the storage unit 23 to perform processing according to the programs.

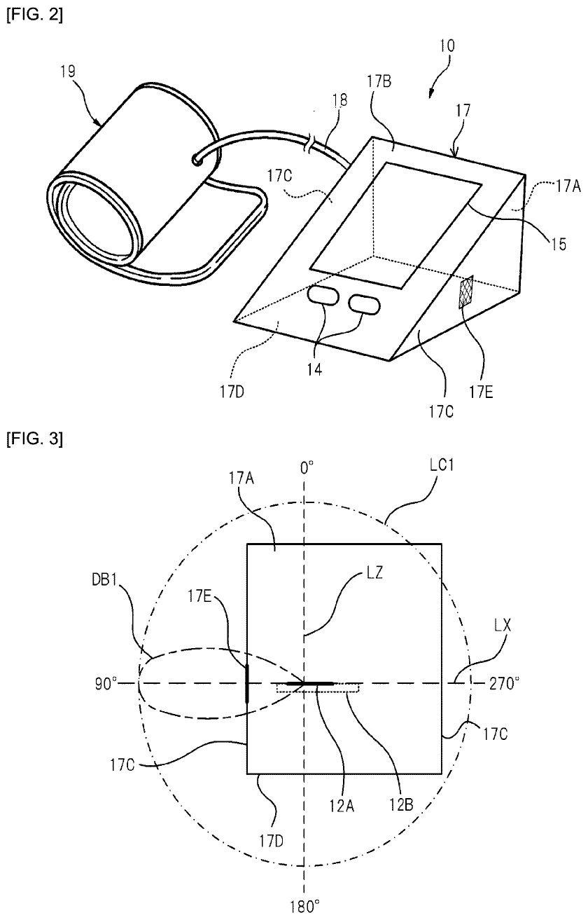

FIG. 2 is a schematic diagram illustrating an example of an external configuration of the blood pressure monitor 10. The blood pressure monitor 10 includes a housing 17 that accommodates at least the processor 11, the communication unit 12, and the storage unit 13 that are illustrated in FIG. 1, a cuff 19 that can be wound around a measurement site (for example, an upper arm) of a user, and an air tube 18 that connects the housing 17 and the cuff 19.

The housing 17 is a pentahedron in the example of FIG. 2, and is placed on a placement surface such as a desk or a floor when the blood pressure monitor 10 is used (when blood pressure information is measured). The outer surfaces of the housing 17 include a bottom surface 17D facing the placement surface when the blood pressure monitor 10 is used, an upper surface 17B forming a predetermined angle with the bottom surface 17D, a pair of side surfaces 17C substantially perpendicular to the bottom surface 17D, and a rear surface 17A. The display section 15 and the operating section 14 are provided on the upper surface 17B.

An information display section 17E from which first information can be read by imaging is provided on one side surface 17C of the housing 17. The information display section 17E is a region in which a text such as numbers, characters, or symbols representing the first information, or a code image such as a one dimensional code or a two dimensional code representing the first information is printed or attached with an attachment member such as a seal. The first information is information unique to the blood pressure monitor 10, and is, for example, address information of the communication unit 12, individual identification information (such as a serial number) of the blood pressure monitor 10, or the like. The first information may be numbers, characters, symbols, or the like randomly generated for each blood pressure monitor 10. The first information is also stored in the storage unit 13.

In the management system 100, the management application for managing the blood pressure information is installed and stored in advance in the storage unit 23 of the smartphone 20. The management application processes captured image data obtained by imaging the information display section 17E with the imaging unit 26 of the smartphone 20, and thus can acquire the first information by recognizing the text included in a portion of the information display section 17E in the captured image data, or can acquire the first information by decoding the code image of a portion of the information display section 17E in the captured image data.

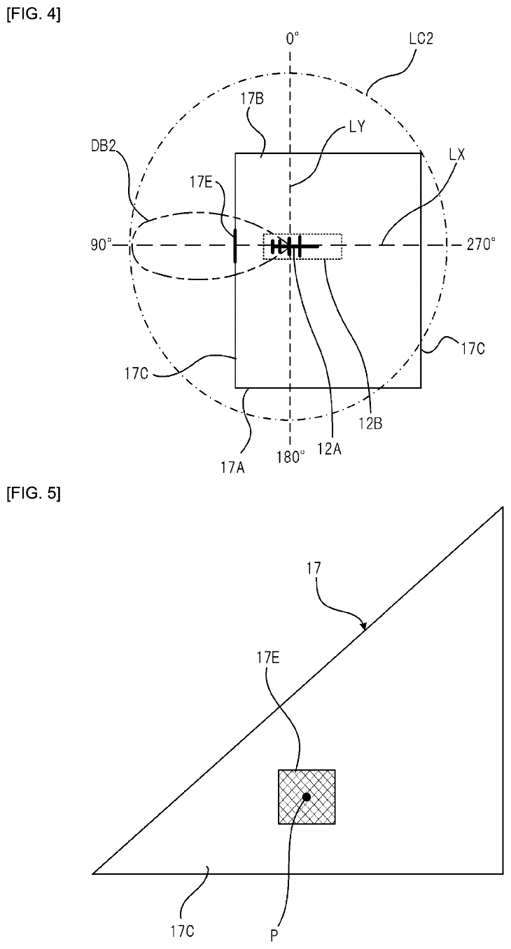

FIG. 3 is a diagram illustrating a schematic configuration of the communication unit 12, and is a schematic diagram of the housing 17 as viewed from the rear surface 17A side. FIG. 4 is a diagram illustrating the schematic configuration of the communication unit 12, and is a schematic diagram of the housing 17 as viewed from the upper surface 17B side. In FIG. 4, the display section 15 and the operating section 14 are omitted. The communication unit 12 has a configuration in which an antenna 12A is formed as a conductive wire pattern on the surface of a flat substrate 12B. In the example in the drawing, the antenna 12A is formed of a Yagi antenna pattern.

In the example of FIGS. 3 and 4, the surface of the substrate 12B of the communication unit 12 is parallel to the bottom surface 17D. FIG. 3 illustrates a virtual axis LZ passing through a feed unit of the antenna 12A and perpendicular to the substrate 12B, a virtual axis LX passing through the feed unit of the antenna 12A and parallel to the substrate 12B, and a virtual circle LC1 centered at the feed unit of the antenna 12A. FIG. 4 illustrates a virtual axis LY perpendicular to the virtual axes LX and LZ and passing through the feed unit of the antenna 12A, and a virtual circle LC2 centered at the feed unit of the antenna 12A. Radiation characteristics of the antenna 12A are represented by a radiation pattern DB1 on a plane including the virtual axes LX and LZ illustrated in in FIG. 3 and a radiation pattern DB2 on a plane including the virtual axes LX and LY illustrated in FIG. 4. The radiation pattern DB1 and the radiation pattern DB2 are schematically illustrated. The virtual circle LC1 and the virtual circle LC2 each indicate a position in which the directivity (gain) is largest.

In FIG. 3, an angle formed by a straight line connecting the feed unit of the antenna 12A and one point on the virtual circle LC1 and a portion of the virtual axis LZ, which is located above the feed unit is defined as a direction with respect to the antenna 12A. In FIG. 4, an angle formed by a straight line connecting the feed unit of the antenna 12A and one point on the virtual circle LC2 and a portion of the virtual axis LY, which is located above the feed unit is defined as a direction with respect to the antenna 12A.

As illustrated in FIGS. 3 and 4, the antenna 12A has directivity in a 90 degree direction (the first direction), which is larger than directivity in a direction other than the 90 degree direction (the second direction) and is largest in all directions. The antenna 12A is configured such that the directivity in directions within the main beam width of 90 degrees and the vicinity thereof (for example, respective angles from 60 degrees to 120 degrees) is larger than the directivity in all directions other than these directions by a threshold or greater (for example, twice or more). In other words, the antenna 12A has the strong directivity in the first direction.

As illustrated in FIGS. 3 and 4, when virtual lines (corresponding to a portion of the virtual axis LX, which is located on the left side from the virtual axis LZ illustrated in FIG. 3 and a portion of the virtual axis LX, which is located on the left side from the virtual axis LY illustrated in FIG. 4) extending from the feed unit of the antenna 12A in the 90 degree direction are set, the virtual lines and the side surface 17C intersect with each other.

FIG. 5 is a diagram of the housing 17 of the blood pressure monitor 10 as viewed from the side surface 17C side on which the information display section 17E is provided. As illustrated in FIG. 5, an intersection P with the virtual line above is present on the side surface 17C. The information display section 17E is disposed near the intersection P. As illustrated in FIG. 5, the information display section 17E is preferably overlapped with the intersection P, but is not necessarily overlapped with the intersection P. In the example of FIG. 5, the information display section 17E has a rectangular shape, and the center thereof coincides with the intersection P.

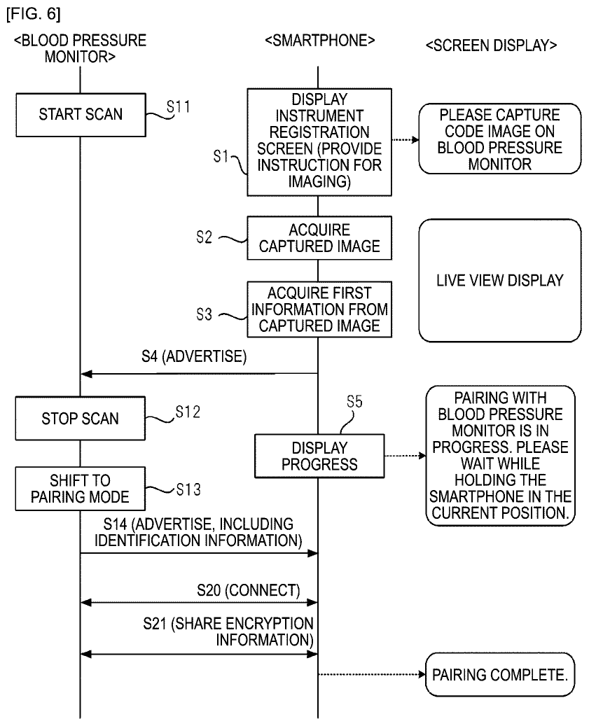

Method of Pairing Between Blood Pressure Monitor and Smartphone Next, a method of pairing between the blood pressure monitor 10 and the smartphone 20 will be described. FIG. 6 is a sequence chart illustrating the method of pairing between the blood pressure monitor 10 and the smartphone 20.

The user powers on the blood pressure monitor 10 and operates the smartphone 20 to activate the management application. The processor 11 of the blood pressure monitor 10 activated allows the communication unit 12 to start scanning (step S11). Scanning means that the communication unit 12 is brought into a reception state to acquire information of surrounding devices. When the management application is activated, the processor 21 of the smartphone 20 allows the display section 25 to display an instrument registration screen (step S1). In step S1, the processor 21 of the smartphone 20 allows the display section 25 to display the instrument registration screen including information providing an instruction to image the information display section 17E of the blood pressure monitor 10. The processor 21 allows the communication unit 22 to start scanning in conjunction with step S1.

As illustrated in FIG. 6, for example, the message “Please capture the code image on the blood pressure monitor” is displayed in the instrument registration screen displayed in step S1. The message may be output via voice. When the user acknowledges the message, the user brings the imaging unit 26 of the smartphone 20 close to the information display section 17E and performs an operation of imaging the information display section 17E. In accordance with this operation, the processor 21 of the smartphone 20 allows the imaging unit 26 to capture an image and acquires captured image data (denoted as “captured image” in the drawing) (step S2).

The processor 21 of the smartphone 20 processes the acquired captured image data and acquires the first information from the image of the information display section 17E included in the captured image data (step S3). Then, the processor 21 of the smartphone 20 generates an advertisement packet including second information based on the acquired first information, and allows the communication unit 22 to start advertisement (step S4). In step S4, the advertisement packet including the second information is periodically broadcast. The second information may be the first information itself, or when the first information is encrypted with a key managed by the management system 100, the second information may be obtained by decrypting the first information with the key.

While the processing from step S2 to step S3 is being performed, the processor 21 of the smartphone 20 controls the display section 25 to display a live view of an image captured by the imaging unit 26. After step S3, the processor 21 of the smartphone 20 controls the display section 25 to display information that indicates the progress of pairing (step S5). The display section 25 displays, for example, the message “Pairing with blood pressure monitor is in progress. Please wait while holding the smartphone in the current position” is displayed.

The processor 11 of the blood pressure monitor 10 receives a broadcast signal transmitted from the smartphone 20 in the processing of step S4, and then when the same first information as that stored in the storage unit 13 is included in the broadcast signal and the received strength of the broadcast signal is at a predetermined threshold or greater, the processor 11 stops scanning by the communication unit 12 (step S12) and shifts to a pairing mode (step S13). The pairing mode is a mode in which processing of sharing, with the information terminal, encryption information for performing encrypted communication by near-field wireless communication can be executed. The blood pressure monitor 10 can be paired with the information terminal only when the blood pressure monitor 10 is operating in the pairing mode. When the received broadcast signal does not include the same information as that stored in the storage unit 13 or when the received strength is less than the threshold value, the processor 11 of the blood pressure monitor 10 continues scanning without shifting to the pairing mode.

In a state where the processing from step S2 to step S4 is being performed, the smartphone 20 is located at a position substantially opposed to the side surface 17C of the blood pressure monitor 10, and is located at a position close to the information display section 17E to the extent that the first information can be acquired from the information display section 17E. In other words, the smartphone 20 is located in a range in which the directivity of the communication unit 12 of the blood pressure monitor 10 is strong. Therefore, even in the present embodiment in which the antenna 12A of the communication unit 12 has directivity, by imaging the information display section 17E with the smartphone 20 and holding the state thereof, the blood pressure monitor 10 can be shifted to the pairing mode without performing a special operation on the blood pressure monitor 10.

Note that, by disposing the information display section 17E at a location near the intersection P such that the center of the information display section 17E is within the range of the main lobe 3-dB beam width (maximum angle of 80 degrees) projected from the antenna 12A onto the side surface 17C, the smartphone 20 can be more accurately guided to the range in which the directivity of the communication unit 12 is strong, and the blood pressure monitor 10 can be more reliably shifted to the pairing mode. In addition, by setting the size of the information display section 17E (a diameter of a circumscribed circle in the case of a polygonal shape of a three-sided or more polygon, or a diameter in the case of a circular shape) to be 1 cm or more and 5 cm or less, the distance between the information display section 17E and the smartphone 20 (in other words, the distance between the communication unit 12 and the smartphone 20) when reading the first information from the information display section 17E can be sufficiently reduced. Therefore, the blood pressure monitor 10 can be reliably shifted to the pairing mode by the smartphone 20 present near the blood pressure monitor 10.

When shifting to the pairing mode, the processor 11 of the blood pressure monitor 10 allows the communication unit 12 to start advertisement (step S14). In step S14, a packet including information (for example, individual identification information of the blood pressure monitor 10 or the like) necessary for establishing a connection for communication by near-field wireless communication is periodically broadcast. This information may be shared with the first information.

When the processor 21 of the smartphone 20 acquires a broadcast signal transmitted from the blood pressure monitor 10 in the processing of step S14, the processor 21 issues a connection request to the blood pressure monitor 10 via the communication unit 22 if the received strength of the broadcast signal is at a threshold or greater. When the processor 11 of the blood pressure monitor 10 responds to the connection request via the communication unit 12, a connection for communication by near-field wireless communication is established between the blood pressure monitor 10 and the smartphone 20 (step S20). Here, since the blood pressure monitor 10 and the smartphone 20 are located close to each other, the connection therebetween is established in step S20.

In a state where the processing from step S2 to step S5 and the processing from step S12 to step S14 are being performed, the smartphone 20 is at a position in which the smartphone 20 can image the information display section 17E. Therefore, the smartphone 20 is located in the range in which the directivity of the communication unit 12 of the blood pressure monitor 10 is strong. Consequently, even in the present embodiment in which the antenna 12A of the communication unit 12 has directivity, communication between the blood pressure monitor 10 and the smartphone 20 can be stably performed.

When a connection for communication between the smartphone 20 and the blood pressure monitor 10 is established, mutual authentication between the smartphone 20 and the blood pressure monitor 10 is performed by a predetermined method, and when the mutual authentication is completed, encryption information (for example, an encryption key or the like) for performing encrypted communication by near-field wireless communication is shared between the smartphone 20 and the blood pressure monitor 10 (step S21). The sharing of encryption information means that the processor 11 of the blood pressure monitor 10 generates encryption information, stores the encryption information in the storage unit 13, and transmits the encryption information to the smartphone 20, and the smartphone 20 stores the encryption information in the storage unit 23. The processor 21 of the smartphone 20 may generate encryption information, store the encryption information in the storage unit 23, and transmit the encryption information to the blood pressure monitor 10, and the blood pressure monitor 10 may store the encryption information in the storage unit 13 to share the encryption information.

After step S21, the processor 21 of the smartphone 20 allows the display section 25 to display, for example, a message indicating that pairing has been completed. Afterward, the processor 21 of the smartphone 20 disconnects the connection for communication with the blood pressure monitor 10. Thereafter, secure communication using the encryption information can be established between the smartphone 20 and the blood pressure monitor 10. For example, when the measurement start button 14A is pressed in a state in which the cuff is wrapped around the arm or the wrist, blood pressure information is derived and stored in the storage unit 13. Then, the processor 11 of the blood pressure monitor 10 activates the communication unit 12, establishes a connection with the smartphone 20 with which the encryption information has been shared, encrypts the blood pressure information, and transmits the encrypted blood pressure information to the smartphone 20.

It is preferable that the processor 11 of the blood pressure monitor 10 sets the radio signal strength of the communication unit 12 to a first strength in a state before shifting to the pairing mode in step S13 and that the processor 11 sets the radio signal strength of the communication unit 12 to a second strength lower than the first strength in a state after shifting to the pairing mode. Thus, when a different information terminal located at a position more or less separated from the blood pressure monitor 10 and located on the virtual line described above, the radio signal strength from the blood pressure monitor 10 becomes weak in the different information terminal. As a result, connection establishment between the different information terminal and the blood pressure monitor 10 can be prevented. This makes it possible to achieve pairing with the intended smartphone 20 located near the blood pressure monitor 10.

As described above, according to the blood pressure monitor 10, only the smartphone 20 that can image the information display section 17E near the blood pressure monitor 10 enables the blood pressure monitor 10 to shift to a state in which pairing can be performed. Therefore, the blood pressure monitor 10 can be prevented from being paired with an unintended information terminal located away from the blood pressure monitor 10.

In the blood pressure monitor 10, the information display section 17E is formed of a printed region or a sticking member, but is not limited thereto. For example, the display section 15 can function as the information display section 17E. In this case, the processor 11 displays the first information (text or code image) in a partial range of the display section 15. In the blood pressure monitor 10, the above-described virtual lines of the antenna 12A of the communication unit 12 are arranged to intersect with each other in the above-described range of the display section 15 on the upper surface 17B. Thus, when the blood pressure monitor 10 is powered on and the first information displayed on the display section 15 is imaged by the smartphone 20, the blood pressure monitor 10 shifts to the pairing mode, and then pairing is performed between the blood pressure monitor 10 and the smartphone 20. According to this configuration, the first information can be displayed as long as the display section 15 is operating, and thus, a lack of the first information can be prevented.

In addition, although the example in which the antenna 12A is formed of the Yagi antenna pattern is described, the antenna 12A may be an antenna having any pattern as long as the antenna has a radiation pattern with strong directivity in a specific direction, and other patterns may be adopted.

Although various embodiments are described above, it will be obvious that the present invention is not limited to such examples. It will be apparent to those skilled in the art that various changes and modifications can be made within the scope of the claims, and it will be understood that these naturally belong to the technical scope of the present invention. In addition, components of the above-described embodiments may be combined as appropriate without departing from the spirit of the invention.

The present application is based on Japanese Patent Application (Japanese Patent Application No. 2023-093953) filed on Jun. 7, 2023, the contents of which are incorporated herein by reference.

### REFERENCE NUMERALS LIST

10 Blood pressure monitor
11, 21 Processor
12, 22 Communication unit
12A Antenna
12B Substrate
13, 23 Storage unit
14, 24 Operating section
14A Measurement start button
14B Communication button
15, 25 Display section
16 Sensor unit
17 Housing
17A Rear surface
17B Upper surface
17C Side surface
17D Bottom surface
17E Information display section
18 Air tube
19 Cuff
20 Smartphone
26 Imaging unit
100 Management system
LC1, LC2 Virtual circle
DB1, DB2 Radiation pattern

What is claimed is:

1. A measuring device, comprising:
a near-field wireless communication unit configured to perform communication by a communication method that enables pairing;
a processor; and
a housing adapted to accommodate the processor and the near-field wireless communication unit, wherein
an antenna of the near-field wireless communication unit has directivity in a first direction that is larger than directivity in a second direction other than the first direction and is largest,
an information display section from which first information can be acquired by imaging is provided on a surface among outer surfaces of the housing, the surface including an intersection with a virtual line extending from the antenna in the first direction, and
when the processor receives a signal including information based on the first information and a received strength of the signal is at a threshold or greater, the processor starts processing necessary for pairing.

2. The measuring device according to claim 1, wherein
when the processor receives the signal and the received strength of the signal is at the threshold or greater, the processor starts transmission of a broadcast signal including information necessary to establish a connection for communication by near-field wireless communication, establishes a connection with an information terminal that has responded to the broadcast signal, and performs pairing.

3. The measuring device according to claim 2, wherein
when the processor receives the signal and the received strength of the signal is at the threshold or greater, the processor reduces a radio signal strength of the antenna to be lower than a radio signal strength at a timing before the signal is received.

4. The measuring device according to claim 1, wherein
the information display section is a display on which the first information can be displayed, and
the display is configured to display a measurement result.

5. The measuring device according to claim 2, wherein
the information display section is a display on which the first information can be displayed, and
the display is configured to display a measurement result.

6. The measuring device according to claim 3, wherein
the information display section is a display on which the first information can be displayed, and
the display is configured to display a measurement result.
